(19) United States
(12) Patent Application Publication
(10) Pub. No.: US 2016/0192677 A1
(43) Pub. Date: Jul. 7, 2016

(54) METHOD OF TREATING CORAL, A CORAL FOOD COMPOSITION AND A METHOD OF MANUFACTURE

(71) Applicants: Peter Saris, Magna, UT (US); Kyle Clark Howes, Herriman, UT (US); Jeremy James Olsen, Sandy, UT (US)

(72) Inventors: Peter Saris, Magna, UT (US); Kyle Clark Howes, Herriman, UT (US); Jeremy James Olsen, Sandy, UT (US)

(21) Appl. No.: 14/959,516

(22) Filed: Dec. 4, 2015

Related U.S. Application Data
(60) Provisional application No. 62/087,486, filed on Dec. 4, 2014.

(51) Int. Cl. A23L 1/10; A61K 35/742; A61K 38/17; A61K 35/741; A61K 35/747; A23L 1/18

(52) U.S. Cl. CPC A23K 1/007; A61K 35/741; A61K 35/742; A61K 35/747; A61K 38/1706; A61K 38/1709; A23K 1/009; A23K 1/103; A23K 1/1603; A23K 1/175; A23K 1/1631; A61K 2035/115

(57) ABSTRACT

There is a coral food composition and the method of manufacture thereof, including a quantity of protein from a sea-based source and a quantity of probiotics from a land-based source; or the coral food composition includes a quantity of protein from a land-based source and a quantity of probiotics from a sea-based source. The quantity of probiotics is a fermented land-based organism. The quantity of protein is fermented. The food composition includes a quantity of one or more of the following: microalgae, plankton, fish meal, shrimp, crustacean, and algae. The food composition includes a quantity of vitamins and minerals. The food composition includes a quantity of probiotics.

### CROSS-REFERENCE TO RELATED APPLICATIONS

This invention claims priority, under 35 U.S.C. §120, to the U.S. Provisional Patent Application No. 62/087,486 by Peter Saris et al. filed on Dec. 4, 2014, which is incorporated by reference herein in its entirety.

### BACKGROUND OF THE INVENTION

1. Field of the Invention

The present invention relates to methods of treating coral and food compositions, specifically a coral food composition and their methods of manufacture.

2. Description of the Related Art

Coral are marine invertebrates that live in compact colonies of groups of polyps. Hermatypic corals have a symbiotic relationship with photosynthetic microalgae and secrete calcium carbonate to form a hard skeleton. Coral is notoriously difficult to keep healthy (especially hard corals) and especially difficult, often impossible to revive when it becomes deadened or otherwise in poor health. In nature, coral form reefs, which are home to extremely diverse and high density populations of plants and animals.

Coral reefs are under stress worldwide due to many factors, including but not limited to pollution, coral mining, overfishing, destructive fishing practices, construction, changes in climate, etc. In some parts of the world most of the existing reefs are endangered and some estimate that over 50% of the world's coral reefs will be destroyed by 2030.

Coral is used is saltwater fishkeeping and in some cases (e.g. reef tanks) is the primary organism within the tank. Soft coral is easier to keep and care for, so it tends to be more popular. Hard coral (small polyp stony coral) requires much greater care and attention and therefore is generally kept by more serious enthusiasts.

Coral may be used for a variety of purposes, including but not limited to jewelry, medicine, construction materials, and climate research. Accordingly, commercial and/or scientific coral farming is used to cultivate coral and/or to restore coral reefs that may have been damages or are under decline. Accordingly, there is a substantial need to promote the health and well-being of coral of various types, especially the more difficult to care for hard corals, and/or to promote the health and well-being of other organisms.

Some improvements have been made in the field. Examples of references related to the present invention are described below in their own words, and the supporting teachings of each reference are incorporated by reference herein:

U.S. Pat. No. 4,741,904, issued to Smith et al., discloses a composition useful as fish and crustacean feed consisting essentially of (a) from about 0.5 to 10 weight percent of a water insoluble polymer having a melting point below about 110.degree. C., selected from the group consisting of polyamides and copolymers of ethylene with from about 15 to about 45 weight percent of at least one ethylenically unsaturated comonomer; (b) from about 75 to about 95 weight percent of a nutrient medium selected from the group consisting of fish meal, crustacea meal, grain derived products, plant derived products, animal derived products, and fish by-products; (c) from 0 to about 20 weight percent of a lubricant selected from the group consisting of edible oil and fish solid solubles; (d) from 0 to about 10 weight percent of a vitamin and mineral concentrate; (e) from 0 to about 10 weight percent of a preservative.

U.S. Pat. No. 5,047,250, issued to Prieels et al., discloses a method of feeding fry, shellfish or mollusks comprising directly feeding the fry, shellfish or mullusks a dried yeast feed of enhanced nutritive value comprising active yeast and up to, but not exceeding, 20% by dry weight of fish oil.

U.S. Pat. No. 6,645,536, issued to D'Abramo, discloses a formulated, microbound diet product for the culture of larval fish and crustaceans either in a dry or moist form is disclosed. The food product contains protein sources such as fish protein hydrosylate, casein, egg yolk, binding agents such as soy lecithin, wheat gluten, and alginate. Other ingredients such as vitamins and minerals, lipid sources, carbohydrate sources, pigment sources, and attractant compounds are included in the diet for nutritional completeness. A method for preparation of the food product is also disclosed.

U.S. Pat. No. 8,198,067, issued to Kyle, discloses a microbial biomass, made from algae, bacteria, fungi, yeast, or combinations thereof, provides a feed for animals raised either in agriculture or aquaculture. A feed additive, and a therapeutic composition can also be made from a microbial biomass of algae, bacteria, fungi, yeast, or combinations thereof. The feed, feed additive, and therapeutic composition can comprise one or more proteins, peptides, antibodies, antibody fragments, or a combination thereof, wherein said proteins, peptides, antibodies, antibody fragments, or a combination thereof are non-native to the microbes of the biomass. The biomass can have therapeutic, bioactive, nutritional, and/or immunogenic properties.

U.S. Patent Application Publication No.: 2011/0189365, by Tagrin, discloses a method for preserving marine aquarium foodstuffs such as zooplankton crustaceans, gelatinous organisms, vertebrate and invertebrate larvae and eggs, shellfish, mollusks, fish and fish roe, oysters and clams, and sea urchins, wherein a supersaturated saline solution is prepared from reverse osmosis deionized water and marine salt, said solution is chilled to a low temperature, and said foodstuffs are immersed into said solution such that moisture is withdrawn from said foodstuffs via osmosis. The resulting product may then be stored and shipped at low temperatures for subsequent rehydration and use as a food product for a salt water aquarium ecosystem.

The inventions heretofore known suffer from a number of disadvantages which include being limited in use, being inefficient, being ineffective, being expensive, being unduly complex, being difficult to manufacture, being unable to facilitate organism/colony growth, not providing color enhancement, not accelerating organism/colony growth, not repairing coral colonies, failing to bolster immune systems of an organism/colony, and failing to revive deadened coral.

What is needed is a coral food composition that solves one or more of the problems described herein and/or one or more problems that may come to the attention of one skilled in the art upon becoming familiar with this specification.

### SUMMARY OF THE INVENTION

The present invention has been developed in response to the present state of the art, and in particular, in response to the problems and needs in the art that have not yet been fully solved by currently available coral food compositions. Accordingly, the present invention has been developed to provide an effective and efficient coral food composition.

According to one embodiment of the invention, there is a coral food composition that may include a quantity of protein from a sea-based source. The coral food composition may include a quantity of probiotics from a land-based source that may be in functional communication with the quantity of protein.

According to one embodiment of the invention, there is a coral food composition that may include a quantity of protein from a land-based source. The coral food composition may include a quantity of probiotics from a sea-based source that may be in functional communication with the quantity of protein.

The food composition may be a powder. The quantity of protein may include both land and sea-based proteins. The quantity of probiotics may be a fermented land-based organism. The food composition may be a paste. The quantity of protein may be fermented. The food composition may include a quantity of one or more of the following: microalgae, plankton, fish meal, shrimp, crustacean, and algae. The food composition may include a quantity of vitamins and minerals. The vitamins and minerals may be from land-based plants. The food composition may include a quantity of probiotics. The probiotics may include non-digestible fiber. It may be that the probiotic quantity includes bacteria species selected from the group of bacteria species consisting of Lactobacillus, Saccharomyces, and Baccillus; and/or that the quantity of protein may be a sea-based protein selected from the group of sea-based proteins consisting of: fish, and crustacean.

According to one embodiment of the invention, there is a method of manufacturing a coral food composition. The method may include the step of providing a quantity of protein from a sea-based source or a land-based source. The method may include the step of providing a quantity of probiotics from a sea-based source or a land-based source but not the same, as the quantity of protein. The method of manufacturing a coral food composition may include the step of fermenting the quantity of protein with the quantity of probiotics thereby forming a fermented mixture. The method may include the step of converting the fermented mixture into a powder or paste.

The quantity of protein may be both land-based and sea-based proteins. The quantity of probiotics may be a fermented land-based organism. The method of manufacturing a coral food composition may include the step of providing a quantity of one or more of the following: microalgae, plankton, fish meal, shrimp, crustacean, and algae. The method may include the step of providing a quantity of vitamins and minerals. The vitamins and minerals may be from land-based plants. The method may include the step of providing a quantity of probiotics. The probiotics may include non-digestible fiber.

According to one embodiment of the invention, there may be a method of treating coral. The method may include the step of applying a food composition to the coral. The food composition may include a quantity of protein from a sea-based source. The food composition may include a quantity of probiotics from a land-based source. The food composition may include a quantity of protein from a land-based source. The food composition may include a quantity of probiotics from a sea-based source.

The food composition may be a powder. The quantity of protein may include both land and sea-based proteins. The quantity of probiotics may be a fermented land-based organism. The food composition may be a paste. The quantity of protein may be fermented. The method of treating coral may include the step of providing a quantity of one or more of the following: microalgae, plankton, fish meal, shrimp, crustacean, and algae. The method may include the step of providing a quantity of vitamins and minerals. The vitamins and minerals may be from land-based plants. The method may include the step of providing a quantity of probiotics. The probiotics may include non-digestible fiber.

Reference throughout this specification to features, advantages, or similar language does not imply that all of the features and advantages that may be realized with the present invention should be or are in any single embodiment of the invention. Rather, language referring to the features and advantages is understood to mean that a specific feature, advantage, or characteristic described in connection with an embodiment is included in at least one embodiment of the present invention. Thus, discussion of the features and advantages, and similar language, throughout this specification may, but do not necessarily, refer to the same embodiment.

Furthermore, the described features, advantages, and characteristics of the invention may be combined in any suitable manner in one or more embodiments. One skilled in the relevant art will recognize that the invention can be practiced without one or more of the specific features or advantages of a particular embodiment. In other instances, additional features and advantages may be recognized in certain embodiments that may not be present in all embodiments of the invention.

These features and advantages of the present invention will become more fully apparent from the following description and appended claims, or may be learned by the practice of the invention as set forth hereinafter.

### BRIEF DESCRIPTION OF THE DRAWINGS

In order for the advantages of the invention to be readily understood, a more particular description of the invention briefly described above will be rendered by reference to specific embodiments that are illustrated in the appended drawing(s). It is noted that the drawings of the invention are not to scale. The drawings are mere schematics representations, not intended to portray specific parameters of the invention. Understanding that these drawing(s) depict only typical embodiments of the invention and are not, therefore, to be considered to be limiting its scope, the invention will be described and explained with additional specificity and detail through the use of the accompanying drawing(s), in which:

FIG. 1 is a method of manufacturing a coral food composition, according to one embodiment of the invention; and

FIG. 2 is a method of treating coral, according to one embodiment of the invention.

### DETAILED DESCRIPTION OF THE INVENTION

For the purposes of promoting an understanding of the principles of the invention, reference will now be made to the exemplary embodiments illustrated in the drawing(s), and specific language will be used to describe the same. It will nevertheless be understood that no limitation of the scope of the invention is thereby intended. Any alterations and further modifications of the inventive features illustrated herein, and any additional applications of the principles of the invention as illustrated herein, which would occur to one skilled in the relevant art and having possession of this disclosure, are to be considered within the scope of the invention.

Reference throughout this specification to an “embodiment,” an “example” or similar language means that a particular feature, structure, characteristic, or combinations thereof described in connection with the embodiment is included in at least one embodiment of the present invention. Thus, appearances of the phrases an “embodiment,” an “example,” and similar language throughout this specification may, but do not necessarily, all refer to the same embodiment, to different embodiments, or to one or more of the figures. Additionally, reference to the wording “embodiment,” “example” or the like, for two or more features, elements, etc. does not mean that the features are necessarily related, dissimilar, the same, etc.

Each statement of an embodiment, or example, is to be considered independent of any other statement of an embodiment despite any use of similar or identical language characterizing each embodiment. Therefore, where one embodiment is identified as “another embodiment,” the identified embodiment is independent of any other embodiments characterized by the language “another embodiment.” The features, functions, and the like described herein are considered to be able to be combined in whole or in part one with another as the claims and/or art may direct, either directly or indirectly, implicitly or explicitly.

As used herein, “comprising,” “including,” “containing,” “is,” “are,” “characterized by,” and grammatical equivalents thereof are inclusive or open-ended terms that do not exclude additional unrecited elements or method steps. “Comprising” is to be interpreted as including the more restrictive terms “consisting of” and “consisting essentially of.”

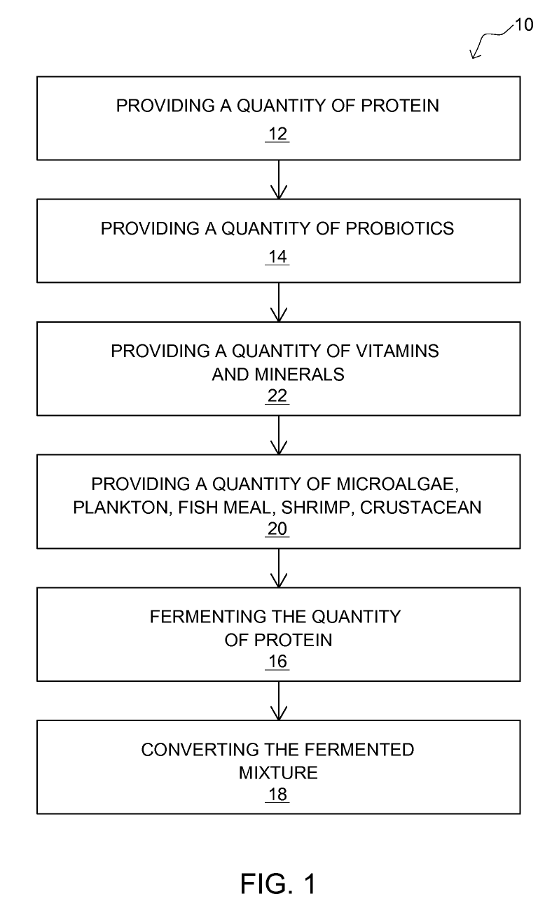

FIG. 1 is a method of manufacturing a coral food composition, according to one embodiment of the invention. There is shown the method of manufacturing a coral food composition 10, specifically the steps of converting a fermented mixture of various specific ingredients into a paste or powder 18.

The illustrated method synergistically combines proteins from one source with probiotics from another source, different from the source of proteins, the sources being land and sea. It is believed that this combination generates beneficial nutrients and/or effects, such as but not limited to generating healthy colonies of powerfully competitive microorganism within the coral itself, not otherwise generated without this artificial combination, as such sources are isolated from each other. It has been observed that this combination has a surprisingly beneficial positive effect on coral and may also have similar such effects on other organisms. In particular, it has been observed that in hard coral that is believed to be essentially dead and beyond recovery by previously known methods has been revived to full health and outstanding color by application of the described coral food composition and that are not revived by application of any of the various ingredients alone. Further, it generally takes months to prepare a coral tank for live coral. It has been observed that the coral food composition(s) described herein allow for a coral tank to be prepared and ready for live coral in mere weeks, thus making it much easier and faster to be able to set up a coral tank. Accordingly, the described composition provides heretofore unknown benefits related to coral keeping and aquaculture and may also be a potent resource in fighting the damaging effects of climate change.

The illustrated method of manufacturing a coral food composition 10 is configured to enhance the health and/or increase the growth of coral. The method 10 includes the step of providing a quantity of protein from a sea-based source or a land-based source 12, such as but not limited to the meat of land and/or sea-based animals (e.g. fish, mollusk, chicken, beef, squid, shrimp). The proteins may be one or both land-based and sea-based proteins.

Proteins are large biomolecules, or macromolecules, consisting of one or more long chains of amino acid residues. Proteins perform a vast array of functions within living organisms, including catalyzing metabolic reactions, DNA replication, responding to stimuli, and transporting molecules from one location to another. Proteins differ from one another primarily in their sequence of amino acids, which is dictated by the nucleotide sequence of their genes, and which usually results in protein folding into a specific three-dimensional structure that determines its activity.

A linear chain of amino acid residues is called a polypeptide. A protein contains at least one long polypeptide. Short polypeptides, containing less than 20-30 residues, are rarely considered to be proteins and are commonly called peptides, or sometimes oligopeptides. The individual amino acid residues are bonded together by peptide bonds and adjacent amino acid residues. The sequence of amino acid residues in a protein is defined by the sequence of a gene, which is encoded in the genetic code. In general, the genetic code specifies 20 standard amino acids; however, in certain organisms the genetic code can includes elenocysteine and—in certain archaea—pyrrolysine. Shortly after or even during synthesis, the residues in a protein are often chemically modified by posttranslational modification, which alters the physical and chemical properties, folding, stability, activity, and ultimately, the function of the proteins. Sometimes proteins have non-peptide groups attached, which can be called prosthetic groups or cofactors. Proteins can also work together to achieve a particular function, and they often associate to form stable protein complexes.

Once formed, proteins only exist for a certain period of time and are then degraded and recycled by the cell's machinery through the process of protein turnover. A protein's lifespan is measured in terms of its half-life and covers a wide range. They may exist for minutes or years with an average lifespan of 1-2 days in mammalian cells. Abnormal and or misfolded proteins are degraded more rapidly either due to being targeted for destruction or due to being unstable.

Like other biological macromolecules such as polysaccharides and nucleic acids, proteins are essential parts of organisms and participate in virtually every process within cells. Many proteins are enzymes that catalyze biochemical reactions and are vital to metabolism. Proteins also have structural or mechanical functions, such as actin and myosin in muscle and the proteins in the cytoskeleton, which form a system of scaffolding that maintains cell shape. Other proteins are important in cell signaling, immune responses, cell adhesion, and the cell cycle. Proteins are also necessary in animals' diets, since animals cannot synthesize all the amino acids they need and must obtain essential amino acids from food. Through the process of digestion, animals break down ingested protein into free amino acids that are then used in metabolism.

Proteins may be purified from other cellular components using a variety of techniques such as ultracentrifugation, precipitation, electrophoresis, and chromatography; the advent of genetic engineering has made possible a number of methods to facilitate purification. Methods commonly used to study protein structure and function include immunohistochemistry, site-directed mutagenesis, X-ray crystallography, nuclear magnetic resonance and mass spectrometry.

The method of manufacturing a coral food composition 10 includes the step of providing a quantity of probiotics from a sea-based source or a land-based source but not the same source, i.e. sea or land, as the quantity of protein 14. In the case where both sea and land-based proteins are included, both at least one of sea or land-based probiotics are provided as well. The quantity of probiotics may be a fermenting/fermented organism or material (e.g. organism species such as but not limited to yeasts and bacteria like Lactobacillus, Saccharomyces, and Bacillus; and/or cultured materials such as but not limited to yogurt, kimchi, sauerkraut, pickled meats and/or vegetables). The quantity of probiotics may also include non-digestible fiber.

Probiotics are microorganisms that are believed to provide health benefits when consumed. The term probiotic is currently used to name ingested microorganisms associated with beneficial effects to humans and animals. The term came into more common use after 1980. The introduction of the concept is generally attributed to recipient Élie Metchnikoff, who in 1907 suggested that “the dependence of the intestinal microbes on the food makes it possible to adopt measures to modify the flora in our bodies and to replace the harmful microbes by useful microbes”. A significant expansion of the potential market for probiotics has led to higher requirements for scientific substantiation of putative beneficial effects conferred by the microorganisms. Studies on the medical benefits of probiotics have yet to reveal a cause-effect relationship, and their medical effectiveness has yet to be conclusively proven for most of the studies conducted thus far.

Commonly claimed benefits of probiotics include the decrease of potentially pathogenic gastrointestinal microorganisms, the reduction of gastrointestinal discomfort, the strengthening of the immune system, the improvement of the skin's function, the improvement of bowel regularity, the strengthening of the resistance to cedar pollen allergens, the decrease in body pathogens, the reduction of flatulence and bloating, the protection of DNA, the protection of proteins and lipids from oxidative damage, and the maintaining of individual intestinal microbiota in subjects receiving antibiotic treatment.

The method 10 includes the step of providing a quantity of vitamins and minerals 22. The vitamins and minerals may be from sea and/or land-based plants, such as but not limited to fruits (e.g. blueberries, raspberries, peaches, noni fruit, and bananas), kelp, seaweed, phyto-planktons, blue-green algae, red algae, green algae, and the like etc.

A vitamin is an organic compound and a vital nutrient that an organism requires in limited amounts. An organic chemical compound (or related set of compounds) is called a vitamin when the organism cannot synthesize the compound in sufficient quantities, and it must be obtained through the diet; thus, the term “vitamin” is conditional upon the circumstances and the particular organism. For example, ascorbic acid (one form of vitamin C) is a vitamin for humans, but not for most other animal organisms. Supplementation is important for the treatment of certain health problems, but there is little evidence of nutritional benefit when used by otherwise healthy people.

By convention the term vitamin includes neither other essential nutrients, such as dietary minerals, essential fatty acids, or essential amino acids (which are needed in greater amounts than vitamins) nor the great number of other nutrients that promote health, and are required less often to maintain the health of the organism.^{[4]} Thirteen vitamins are universally recognized at present. Vitamins are classified by their biological and chemical activity, not their structure. Thus, each “vitamin” refers to a number of vitamer compounds that all show the biological activity associated with a particular vitamin. Such a set of chemicals is grouped under an alphabetized vitamin “generic descriptor” title, such as “vitamin A”, which includes the compounds retinal, retinol, and four known carotenoids. Vitamers by definition are convertible to the active form of the vitamin in the body, and are sometimes inter-convertible to one another, as well.

Vitamins have diverse biochemical functions. Some, such as vitamin D, have hormone-like functions as regulators of mineral metabolism, or regulators of cell and tissue growth and differentiation (such as some forms of vitamin A). Others function as antioxidants (e.g., vitamin E and sometimes vitamin C).^{[5]} The largest number of vitamins, the B complex vitamins, functions as precursors for enzyme cofactors, that helps enzymes in their work as catalysts in metabolism. In this role, vitamins may be tightly bound to enzymes as part of prosthetic groups: For example, biotin is part of enzymes involved in making fatty acids. They may also be less tightly bound to enzyme catalysts as coenzymes, detachable molecules that function to carry chemical groups or electrons between molecules. For example, folic acid may carry methyl, formyl, and methylene groups in the cell. Although these roles in assisting enzyme-substrate reactions are vitamins' best-known function, the other vitamin functions are equally important.

Until the mid-1930s, when the first commercial yeast-extract vitamin B complex and semi-synthetic vitamin C supplement tablets were sold, vitamins were obtained solely through food intake, and changes in diet (which, for example, could occur during a particular growing season) usually greatly altered the types and amounts of vitamins ingested. However, vitamins have been produced as commodity chemicals and made widely available as inexpensive semisynthetic and synthetic-source multi-vitamin dietary and food supplements and additives, since the middle of the 20th century. Study of structural activity, function and their role in maintaining health is called vitaminology.

Vitamins are essential for the normal growth and development of a multicellular organism. Using the genetic blueprint inherited from its parents, a fetus begins to develop, at the moment of conception, from the nutrients it absorbs. It requires certain vitamins and minerals to be present at certain times. These nutrients facilitate the chemical reactions that produce among other things, skin, bone, and muscle. If there is serious deficiency in one or more of these nutrients, a child may develop a deficiency disease. Even minor deficiencies may cause permanent damage.

For the most part, vitamins are obtained with food, but a few are obtained by other means. For example, microorganisms in the intestine—commonly known as “gut flora”—produce vitamin K and biotin, while one form of vitamin D is synthesized in the skin with the help of the natural ultraviolet wavelength of sunlight. Humans can produce some vitamins from precursors they consume. Examples include vitamin A, produced from beta carotene, and niacin, from the amino acid tryptophan.

Once growth and development are completed, vitamins remain essential nutrients for the healthy maintenance of the cells, tissues, and organs that make up a multicellular organism; they also enable a multicellular life form to efficiently use chemical energy provided by food it eats, and to help process the proteins, carbohydrates, and fats required for respiration.

Mineral nutrients are the chemical elements required by living organisms, other than the four elements carbon, hydrogen, nitrogen, and oxygen present in common organic molecules. The term “dietary mineral” is archaic, as the substances it refers to are chemical elements rather than actual minerals.

Chemical elements in order of abundance in the human body include the seven major dietary elements calcium, phosphorus, potassium, sulfur, sodium, chlorine, and magnesium Important “trace” or minor dietary elements, necessary for mammalian life, include iron, cobalt, copper, zinc, manganese, molybdenum, iodine, bromine, and selenium.

Over twenty dietary elements are necessary for mammals, and several more for various other types of life. The total number of chemical elements that are absolutely needed is not known for any organism. Ultratrace amounts of some elements (e.g., boron, chromium) are known to clearly have a role but the exact biochemical nature is unknown, and others (e.g. arsenic, silicon) are suspected to have a role in health, but without proof.

Most chemical elements that enter into the dietary physiology of organisms are in the form of simple compounds. Larger chemical compounds of elements need to be broken down for absorption. Plants absorb dissolved elements in soils, which are subsequently picked up by the herbivores that eat them and so on; the elements move up the food chain. Larger organisms may also consume soil (geophagia) and visit salt licks to obtain limiting dietary elements they are unable to acquire through other components of their diet.

Bacteria play an essential role in the weathering of primary elements that results in the release of nutrients for their own nutrition and for the nutrition of others in the ecological food chain. One element, cobalt, is available for use by animals only after having been processed into complicated molecules (e.g., vitamin B12) by bacteria. Scientists are only recently starting to appreciate the magnitude and role that microorganisms have in the global cycling and formation of biominerals.

The method of manufacturing a coral food composition 10 includes the step of providing a quantity of one or more of the following: microalgae, plankton, fish meal, shrimp, crustacean, and algae 20.

The method of manufacturing a coral food composition 10 includes the step of fermenting the quantity of protein with the quantity of probiotics thereby forming a fermented mixture 16.

Fermentation is a metabolic process that converts sugar to acids, gases or alcohol. It occurs in yeast and bacteria, and also in oxygen-starved muscle cells, as in the case of lactic acid fermentation. Fermentation is also used more broadly to refer to the bulk growth of microorganisms on a growth medium, often with the goal of producing a specific chemical product. French microbiologist Louis Pasteur is often remembered for his insights into fermentation and its microbial causes. The science of fermentation is known as zymology.

Fermentation generally takes place in the lack of oxygen (when the electron transport chain is unusable) and becomes the cell's primary means of ATP (energy) production. It turns NADH and pyruvate produced in the glycolysis step into NAD⁺ and various small molecules depending on the type of fermentation (see examples below). In the presence of O₂, NADH and pyruvate are used to generate ATP in respiration. This is called oxidative phosphorylation, and it generates much more ATP than glycolysis alone. For that reason, cells generally benefit from avoiding fermentation when oxygen is available, the exception being obligate anaerobes which cannot tolerate oxygen.

The method 10 includes the step of converting the fermented mixture into a powder or paste 18. It may be that the process of converting the fermented mixture into a powder or paste is limited to not include methods that would substantially kill-off the included probiotics (e.g. limited in temperature ranges, limited in included materials harmful to the included probiotics). Accordingly, the powder and/or paste will generally still include live probiotics.

According to one embodiment of the invention, there is a method of manufacturing a coral food composition includes the step of mixing or blending land-based proteins and sea-based proteins together. Land-based proteins include but are not limited to those that are sourced from milk, eggs, and/or muscle mass from land-based creatures (cows, chickens, sheep, dogs, pigs, and the like and combinations thereof). Sea-based proteins include but are not limited to those that are sourced from milk, eggs, and/or muscle mass from salt-water dwelling creatures (seals, fish, sharks, shrimp, crab, lobster, whales, octopi, squid, and the like and combinations thereof). Proteins sourced from fresh-water dwelling creatures may also be included.

In one non-limiting embodiment, the mixture is blended/ground/mixed/screened/meshed/pulverized/etc. to include particle sizes distributed within a range of between about 3 microns to 3000 microns such that there is a wide variation of particle sizes within that range. Advantageously, the food composition is able to be consumed by organisms (esp. the various components of coral) having different sizes or which are adapted to eat foods of various sizes, thus providing nutrition to both the polyps and the parasitic zooxanthellae in coral colonies having zooxanthellae.

Generally, the amounts of each to be mixed together will be substantially the same but this is not required. In particular, wherein a particular protein is desired to be a primary experience for the organism to be fed that particular protein may be in amounts much greater than the other protein. Accordingly, the organism may believe itself to be ingesting and metabolizing a familiar substance, while simultaneously ingesting additional beneficial materials without experiencing stress associated with eating an unknown substance.

Mixing and/or blending may be accomplished by grounding the materials to a fine paste and then mixing, mixing using a blender or otherwise chopping the materials to a fine structure and then mixing the same. Such mixing is intended to bring such diverse (land vs. sea) materials into close proximity such that they may interact on a chemical level without large portions remaining out of communication with diverse materials so that they remain unaffected by the same. The specific fineness of the chopping/grounding will be determined by the methods of mixing and the time allowed for the materials to interact, especially with regard to probiotic interaction as described herein.

The method includes the step of adding land-based vitamins and minerals to the protein mixture. Such may include but is not limited to plant and animal materials rich in vitamins and/or minerals from land-based plants and animals (organs from cows, pigs, chicken, etc.; fruits; vegetables; beans; and the like and combinations thereof). The following is a list of non-limiting examples of such sources: spinach leaves, blueberries, raw cocoa powder, animal liver, peach, noni, and the like and combinations thereof. Wherein land-based protein sources are used with whole animals (organs and muscle masses) this step may be simultaneously accomplished with the step of providing land-based proteins. One or more of the following vitamins and minerals may be of particular importance to include (from a natural source as described above and/or from an isolated/concentrated man-made process source): boron, iodine, iron, copper, zinc, manganese, magnesium, bromide, cobalt, molybdenum, vanadium, nickel, tin, rubidium, ascorbic acid (vitamin c), thiamine, riboflavin, niacin, choline, B12, Inositol, Arginine, Glutamine, Lysine, Tyrosine, and/or Polyunsaturated Fatty Acids.

The method also includes the step of adding sea-based vitamins and minerals to the blended proteins. Such may include but is not limited to plant and animal materials rich in vitamins and/or minerals from sea-based plants and animals (organs from fish, shark, whales, seals, etc.; algae; kelp; seaweed; phytoplankton; etc.). The following is a non-limiting list of such sources: seaweed; kelp; oily fish; algae; phytoplankton; fish organs and the like and combinations thereof. Wherein land-based protein sources are used with whole animals (organs and muscle masses) this step may be simultaneously accomplished with the step of providing sea-based proteins. One or more of the following vitamins and minerals may be of particular importance to include (from a natural source as described above and/or from an isolated/concentrated man-made process source): boron, iodine, iron, copper, zinc, manganese, bromide, cobalt, molybdenum, vanadium, nickel, tin, rubidium, ascorbic acid (vitamin c), thiamine, riboflavin, niacin, choline, B12, Inositol, Arginine, Glutamine, Lysine, Tyrosine, and/or Polyunsaturated Fatty Acids.

The method further includes the step of adding probiotics to the mixture. Such include live bacteria that confer a health benefit to the host. Generally they are provided in the form of live yogurt cultures, but may also be sourced from fermented foods (e.g. pickled vegetables (land and/or sea, e.g. Kelp Jelly), tempeh, miso, doen jang, kefir, buttermilk, kimchi, pao cai, sauerkraut, soy sauce, and zha cai), and/or yeasts. The following are examples of strains of bacteria that may be included in a probiotic: Bacillus coagulans, Bacillus subtilis, Bacillus sp., Paenibacillus, Escherichia coli, Bifidobacterium animalis, Bifidobacterium longum, Lactobacillus acidophilus, Lactobacillus johnsonii, Lactobacillus plantarum, Lactobacillus reuteri, and Saccharomyces boulardii.

In one non-limiting example, a foundation is used to culture and/or to grow one or more probiotics. Such a foundation may include one or more ingredients that promote the growth of desirable probiotics, inhibit the growth of undesirable probiotics and/or otherwise support the operation of the foundation, such as but not limited to dextrose, protease peptone, yeast extract, sodium acetate, 2-Phenylethyl, Ammonium Citrate, Dipotassium phosphate, magnesium sulfate, manganese sulfate, bromo cresel green, captan and the like and combinations thereof. One non-limiting example of a foundation is the MRS Broth modified, Vegitone sold under the brand Fluka Analytical by Sigma-Aldrich Co., LLC, of St. Louis, Mo., which foundation promotes the health of lactobacillus-type probiotics in favor against others. Wherein such a foundation is also included as an ingredient in (or remains with the probiotic and therefore included therewith when the probiotic is included), the foundation continues to provide its beneficial functions for the mix.

It may be that probiotics associated with land-based hosts are provided. It may be that probiotics associated with sea-based hosts are provided (such as but not limited to those found in the Eco-Balance product by Dr. Tim's Aquatics of www.drtimsaqautics.com). It may be that both are provided. It may be that two different types of probiotics are provided but they are both either land-based or sea-based. Regardless, since nutrition sources (protein and vitamins/minerals) of both land and sea based sources are present in the mixture, probiotic fermentation processes that do not normally occur will occur, since there will be mixed sea and land based nutrient sources combined with probiotic processes. The mixture is generally mixed well and then allowed to rest for a period of time, wherein the probiotic may then act upon the mixture. Since the mixture includes resources not generally available in nature to the probiotic, it is able to convert the available materials into usable resources that would not otherwise be available to the organism for which the mixture is intended to feed. Accordingly, it may accomplish results not otherwise seen from providing the individual components to the same organism. In particular, it is believed that the organism's metabolic functions may more readily accept such nutrients since they more similarly resemble nutrients already accepted. Since the organisms body is accepting such nutrients and since the organism has never had access to those nutrients, the organism has an abundance of resources with which to grow, fight off infections, reproduce and otherwise function at a level higher than that possible on its traditional food sources. Applicant has observed such results in captive coral colonies and such results have been dramatic and repeatable.

Sugars and/or starches, such as but not limited to fruit juices, honey, nectar, and the like may also be added and may help with fermentation processes. It is recommended to avoid processes and/or storage/delivery environments that will kill the live bacteria of the probiotic, such as but not limited to excessive temperatures, exposure to significant quantities of competitive bacteria, extreme pH, pollutants, contaminants, poisons, and the like and combinations thereof.

The mixture is generally to be disposed into a water tank including coral and other ocean/water species, wherein the mixture is configured to provide a nutrient rich food supply thereto for enhanced growth, repair, immune system improvements and enhanced coloration. It is understood that the mixture may be fed to organisms other than coral.

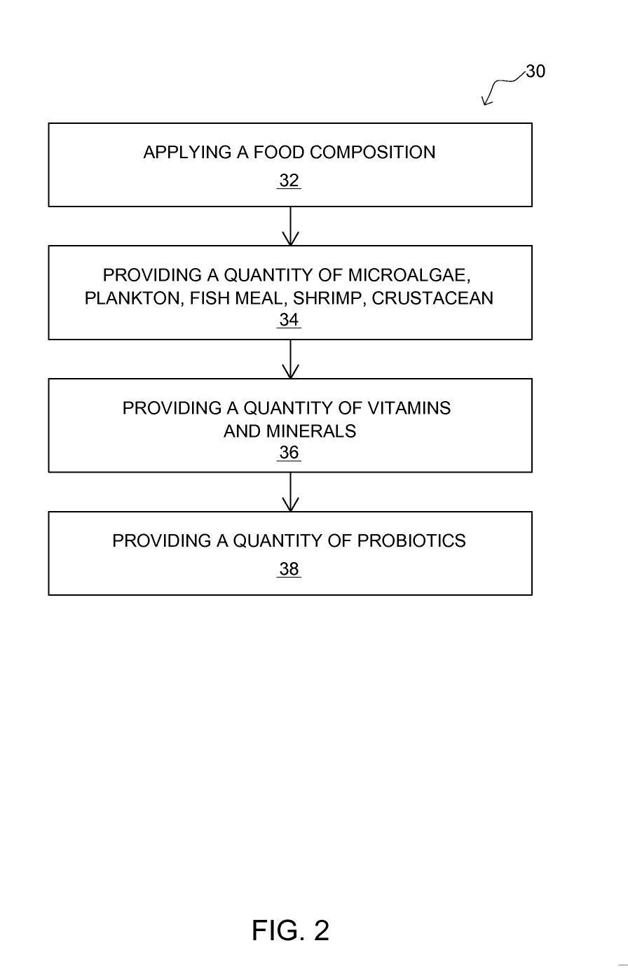

FIG. 2 is a method of treating coral, according to one embodiment of the invention. There is shown a method of treating coral 30, specifically the steps of applying a food composition 32, providing a quantity of vitamins and minerals 36, and providing a quantity of probiotics 38 to coral.

The illustrated method of treating coral 30 to increase growth and improve coral health. The method 30 includes the step of applying a food composition to the coral 32. The food composition includes a quantity of protein from a sea-based source and a quantity of probiotics from a land-based source. Or the food composition includes a quantity of protein from a land-based source and a quantity of probiotics from a sea-based source. The food composition is a powder or a paste. The quantity of protein may be fermented. The quantity of protein may include land and sea-based proteins/protein sources.

The illustrated method of treating coral 30 includes the step of providing a quantity of one or more of the following: microalgae, plankton, fish meal, shrimp, crustacean, and algae 34.

The illustrated method 30 includes the step of providing a quantity of vitamins and minerals 36. The vitamins and minerals are from land-based plants.

The illustrated method of treating coral 30 includes the step of providing a quantity of probiotics. The probiotics include non-digestible fiber. The quantity of probiotics are a fermented land-based organism.

Although corals contain innate or nonspecific immunity systems, they do not produce antibodies and are considered to lack an adaptive immune system. However, recent data demonstrate that corals can develop resistance to specific pathogens and adapt to higher temperatures.

The following is believed to play a role in the beneficial effects of the coral food composition described herein. The coral and zooxanthellae (ZP) have a delicate symbiotic relationship. The ZP are really parasites on the coral that the coral keep in check. The ZP remove the waste products of the coral (carbon dioxide, phosphate, and ammonia for example) and also provide simple sugars (produced from photosynthesis) and other growth factors which the coral have learned to use for growth. When the coral is stressed, either by environmental issues or infection, as a defense mechanism, the coral expel the ZP. This creates a downward spiral for the coral. They grow slower, become more sensitive to infection and if not corrected, die. Probiotic bacteria can protect against infection. A balanced mixture of growth factors as provide by your product may enhance the health of both the coral and the ZP symbionts.

Environmental extremes, such as temperature, pH, water chemistry and sediment can increase the susceptibility of coral to microbial infection and disease. A good example of that often occurs during the warm summer months in the Mediterranean Sea (MS). Warm temperature increases the virulence of Vibrio shiloi. V. Shiloi penetrates the coral's epidermis and produces a toxin which inhibits photosynthesis by ZP and also may kill ZP. During the summer of 2003, coral reefs developed resistance to the pathogen mainly because of a probiotic bacterial species that lysed V. shiloi. See Reshef L et. al. (2006) “The coral probiotic hypothesis”. Environ. Microbiol. 8:2068-2073.

It is generally accepted that probiotic bacteria can help prevent various infections in animals and plants. Live bacteria also have growth factors and vitamins that are assimilated better than the same growth factors and vitamins added as supplements only.

Growing coral requires careful management of numerous environmental factors, any of which if not controlled will create problems. Healthy coral with a good ZP population are able to adjust to minor changes better than coral that are struggling to grow.

In one non-limiting embodiment, there may be a quantity of protein that is sea-based and is fermented using probiotics that are non-native to the sea (e.g. land-based probiotics). The fermentation process allows the probiotics to consume/process the proteins and any bacteria unable to thrive in that nutrient setting are overcome by those who can, thereby forming a probiotic culture that is non-native to aquaculture environments that is able to sustain itself on sea-based proteins. As the probiotic is introduced to the coral, it may then form a colony together with the coral, putting other, especially damaging bacteria and parasites at a disadvantage and/or providing enhanced nutrition to the coral, thus benefiting the coral.

According to one embodiment of the invention, there is a coral food composition for delivery and application of vitamins and minerals from a live source to coral. The coral food composition may include raw cocoa, milk cultured with probiotics (milk that is land-based and milk that is sea-based mixed together), garlic, coconut milk, plus nutrient/food source (insects, algae, fruits, etc.—protein and vitamin/mineral sources).

According to one embodiment of the invention, there is a coral food composition is configured to: improve coral growth, color enhancement, accelerated coral growth, repair coral, bolster immune system of coral, and revive deadened coral.

According to one embodiment of the invention, there is a coral food composition including megavitamin minerals, such as but not limited to: boron, iodine, iron, copper, zinc, manganese, bromide, cobalt, molybdenum, vanadium, nickel, tin, and rubidium. The coral food composition includes megavitamin vitamins such as, but not limited to: ascorbic acid (vitamin c), thiamine, riboflavin, niacin, choline, B12, Inositol, Arginine, Glutamine, Lysine, Tyrosine, and Polyunsaturated Fatty Acids.

Exemplary Recipe #1

According to one embodiment of the invention, there is a coral food composition that may include the following:

10 ml yogurt (greek yogurt mixed with honey yogurt and garlic clove steeped for 3 days)
1 ml ground raw cocoa
½ ml blueberry juice
1 whole full grown zebra danio (type of fish—provides both sea-based protein and sea-based vitamins/minerals)—ground (approx 1 ml)
½ ml raspberry
½ ml blackberry
½ ml megavitamin (e.g. that produced by Aquavitro under the brand name Fuel, 1000 Seachem Drive, Madison Ga. 30650, www.aquavitro.com)
10 ml purified water (by reverse osmosis)
(add coconut milk if the tank magnesium level is dropping)

According to one embodiment of the invention, there is a coral food composition delivery method of the above exemplary recipe that may include shaking a container for 1 minute of coral food composition. Then using a dropper feeding 1 ml for every 30 gallons of tank water twice a day. The coral food composition is to be keep refrigerated, and the yogurt should be kept alive.

Exemplary Recipe #2

Mix together sea-based and land-based milk in substantially equal amounts.

Add substantially equal amounts of raw cocoa powder, ground garlic, coconut milk, ground insects, ground whole shrimp, ground algae, and ground pineapple.

Add a probiotic culture and a powdered megavitamin.

Blend well and store in refrigerator overnight before use.

Exemplary Recipe #3

Mix together: sea-based probiotics with goat milk and spinach, promote fermentation of the same.

Mix together, either separately or together with the above mixture: yogurt including land-based probiotics with anchovies and algae, then promot fermentation of the same.

Combine the two mixtures (if not already combined).

Admixing a fluid-based vitamin supplement and fruit juices into the combined mixture.

Dry and powder the combined mixture by freeze drying.

Admixing powdered garlic and ground raw cocoa, then package.

According to one embodiment of the invention, there is a coral food composition including a quantity of protein from a sea-based source and a quantity of probiotics from a land-based source. Or the coral food composition includes a quantity of protein from a land-based source and a quantity of probiotics from a sea-based source. The food composition is a powder or a paste. The quantity of protein both are land and sea-based proteins. The quantity of probiotics is a fermented land-based organism. The probiotics include non-digestible fiber. The quantity of protein is fermented.

The food composition includes a quantity of one or more of the following: microalgae, plankton, fish meal, shrimp, crustacean, and algae. The food composition includes a quantity of vitamins and minerals. The vitamins and minerals are from land-based plants.

Exemplary Composition #1

Salmon Fish Meal

Freeze Dried Planktons

Brine Shrimp

Rotifers

Copepods

Yeast

Corn Starch

Calcium Powder

Astaxanthin

Spirulina

Garlic Powder

Marine Fish Oil

Soy Flour

Probiotics

With:

a crude protein amount of between about 25% and about 80% by weight

a crude fat amount of between about 4% and about 25% by weight

a crude fiber amount of between about 5% and about 50% by weight

Exemplary Composition #2

Fish Meal

Shrimp

Probiotics

With:

a crude protein amount of between about 25% and about 80% by weight

a crude fat amount of between about 4% and about 25% by weight

a crude fiber amount of between about 5% and about 50% by weight

Exemplary Composition #3

Chicken Meal

Freeze Dried Planktons

Brine Shrimp

Rotifers

Copepods

Astaxanthin

Spirulina

Algae Jelly (e.g. Kelp Jelly: Sea-based Probiotics)

Dried Yogurt (Land-based probiotics)

With:

a crude protein amount of between about 25% and about 80% by weight

a crude fat amount of between about 4% and about 25% by weight

a crude fiber amount of between about 5% and about 50% by weight

Exemplary Method of Applying Coral Food Composition

Mix paste or dry composition with water (generally water from the container/tank holding the coral where it will be applied) in an amount sufficient to generate a fluid slurry. Wait a few minutes and then place the fluid slurry in the container/tank holding the coral. Repeat application 2-3 times a week until desired results are achieved then reduce application frequency (e.g. once per week) to maintain health and growth.

According to one embodiment of the invention, there is a coral food composition that is a compound pro-biotic that natural forms including many naturally occurring vitamins and minerals. This coral composition may be viewed as a bunch of super-foods configured to create the ultimate food for coral and the like. The composition includes a plurality of proteins from land-based sources and from sea-based sources. Land-based sources of protein may be, but not limited to: goat milk, broccoli, spinach, avocados, eggs from ostrich and/or chicken. The land-based proteins may be designed to be mixed with the sea-based proteins. The sea-based proteins may be, but not limited to: spirulina algae, sea milk (mammal milk from the ocean), seaweeds (dolce), kelp, salmon, fish eggs from salmon, rotifer, brine shrimp, artinia shrimp, copapod, and/or fresh water daphnea. The coral food composition may be organically sourced to keep it pure and free from contaminants. The coral food composition includes vitamins and minerals from land-based sources and sea-based sources. The land-based vitamins and minerals may be, but are not limited to: acai berries, blueberries, raspberries, blackberries, peach, and noni. The sea-based sources of vitamins and minerals may be, but are not limited to: kelp, seaweed, phyto-planktons, blue-green algae, red algae, green algae, and the like etc. The coral food composition may also include adding probiotics such as, but not limited to: bacillus (lactobacillus, acilodopolis, etc.) from a yogurt culture. The coral food composition is configured to be a megavitamin supplement for coral. The coral food composition is a delivery system for probiotics and nutrients to coral. The coral food composition may include oatmeal and yeast. The coral food composition generally does not include soy. The coral food may include additional fats, nutrients and/or sugars, including but not limited to raw honey, honey comb, royal jelly, almond milk, coconut milk, pineapple juice, and garlic.

As used herein the term “coral food composition” is not limited to compositions intended for the benefit of coral or only for the benefit of coral, but also includes compositions which may be intended for the benefit of additional or alternative organisms. Such may include one or more of: trans-galactooligosaccharide, inulin, resistant starch, fructooligosaccharide (FOS), lactulose, and/or Mannan Oligosaccharides (MOS). Sources of such may include (and such may be included within a composition as an ingredient therewith): Acacia Gum, Raw Chicory Root, Raw Jerusalem Artichoke, Raw Dandelion Greens, Raw Garlic, Raw Leek, Raw Onion, Cooked Onion, Raw Asparagus, Raw Wheat bran, Whole Wheat flour, Cooked, and/or Raw Banana.

A coral food composition may include pre-biotics, i.e. chemicals which induce growth and/or activity of microorganisms. Often such include non-digestible fiber which acts as a substrate for growth, and/or selectively fermented ingredients that allow specific changes, in composition and/or activity of beneficial microflora.

A coral food composition may include cornstarch. Such may be included in any step of the process, including but not limited to before/after any fermentation, and/or during/after any drying process. Advantageously, cornstarch activates a feeding response from coral and thereby promotes the intake of beneficial materials from the food composition during use.

A coral food composition may be dried and or powdered. Such may be accomplished by one or more steps of evaporation or the like or combinations thereof.

Evaporation steps may include one or more of the following: applying a vacuum (generally partial) to a quantity of composition within a sealed container, heating the composition to a temperature (in combination with pressure) that induces vaporization of water and/or other fluids disposed within the composition; processing the composition through a separator to remove unwanted fluids such as cream, butterfat, oils, and the like; freeze drying (exposing the material to very low temperatures and pressures to induce water removal therefrom); spraying the composition into a superheated (e.g. 325, 350, 375, 400, 425 degrees Fahrenheit) air region (e.g. drying tower) that may be swirling/forced and/or may be treated to have a low humidity in order to maximize heat transfer and water removal; atomizing (using a very fast atomizing wheel) the composition into a superheated (e.g. 325, 350, 375, 400, 425 degrees Fahrenheit) air region (e.g. drying tower) that may be swirling/forced and/or may be treated to have a low humidity in order to maximize heat transfer and water removal.

Once the composition is dried and/or powdered, one may introduce additives to the composition, such as but not limited to vitamins, minerals, amino acids, dried probiotics, dried prebiotics, powdered and/or freeze-dried vegetables/fruits/meats/milks from land and/or sea-based sources, yeast powder, and the like and combinations thereof.

In one non-limiting embodiment, a dry powder composition may be formed by:
mixing together fermentation ingredients, including but not limited to probiotics and materials to be fermented that may be of a different sea vs. land origin from the probiotics (e.g. land-based probiotics and kelp to be thereby fermented), which may also include fermentation supporting ingredients such as but not limited to salts, acids, and the like; then
exposing the mixture to an environment (e.g. heat, pressure, oxygen deprivation) that promotes fermentation for a period of time sufficient to obtain desired amounts of fermentation; then
testing the fermented mixture for the presence/absence of desired/unwanted substances (e.g. specific: acids, proteins, amino acids, microorganisms); then
the fermented mixture is dried and/or powdered (generally in a manner that does not damage the chemical make-up of the fermented mixture as fermentation produces desirable molecules, such manner may include not raising the temperature above a particular point); then
admixing additional dry ingredients that are beneficial but not participatory in the cross-origin fermentation, such as but not limited to vitamins, minerals, dried and powdered plant/animal materials (e.g. powdered raw cocoa, powdered garlic, powdered insects, powdered algae).

Such a powdered composition may be easily stored, shipped and used for beneficial nutrition of organisms, wherein the ingredients selected in the creation thereof may be chosen and/or adapted to the benefit of particular organisms (e.g. coral, fish, cats, dogs, humans, birds). Such would generally be stored in moisture proof containers (e.g. plastic bags) and desiccants may be included therewith. Such advantageously halts microorganism activity and greatly slows degradation of valuable molecules found within the mixture. Accordingly, the stored composition may be conveniently used for a great variety of specific purposes, including but not limited to the revivification of coral.

Advantageously, the methods and/or compositions described herein allow for probiotics to act upon materials not typically acted upon in nature in order to produce nutritive materials that are easy to metabolize, thus delivering new kinds of nutrients that are accessible to both plant and animal based creatures. In the case of hermatypic coral colonies, this provides exceptional food and nutrition to both the plant and animal organisms of the colonies. Since both the plant and animal components of the colonies are able to simultaneously flourish under the same supplement, they do not limit the growth and success of the other as would happen if only one or the other were properly provided enhanced nutrients. Accordingly, dramatic and heretofore unheard of benefits are realized in coral structures using the methods and compositions described herein.

Advantageously, the methods and/or compositions described herein allow for probiotics to act upon materials not typically acted upon in nature in order to produce nutritive materials that are easy to metabolize, thus delivering new kinds of nutrients that are accessible to both plant and animal based creatures. In the case of hermatypic coral colonies, this provides exceptional food and nutrition to both the plant and animal organisms of the colonies. Since both the plant and animal components of the colonies are able to simultaneously flourish under the same supplement, they do not limit the growth and success of the other as would happen if only one or the other were properly provided enhanced nutrients. Accordingly, dramatic and heretofore unheard of benefits are realized in coral structures using the methods and compositions described herein.

It is understood that the above-described embodiments are only illustrative of the application of the principles of the present invention. The present invention may be embodied in other specific forms without departing from its spirit or essential characteristics. The described embodiment is to be considered in all respects only as illustrative and not restrictive. The scope of the invention is, therefore, indicated by the appended claims rather than by the foregoing description. All changes which come within the meaning and range of equivalency of the claims are to be embraced within their scope.

Thus, while the present invention has been fully described above with particularity and detail in connection with what is presently deemed to be the most practical and preferred embodiment of the invention, it will be apparent to those of ordinary skill in the art that numerous modifications, including, but not limited to, variations in size, materials, shape, form, function and manner of operation, assembly and use may be made, without departing from the principles and concepts of the invention as set forth in the claims. Further, it is contemplated that an embodiment may be limited to consist of or to consist essentially of one or more of the features, functions, structures, methods described herein.

What is claimed is:

1. A coral food composition, comprising:
a) a quantity of protein from a sea-based source; and
b) a quantity of probiotics from a land-based source in functional communication with the quantity of protein;
or c) a quantity of protein from a land-based source; and
d) a quantity of probiotics from a sea-based source in functional communication with the quantity of protein.

2. The composition of claim 1, wherein the food composition is a powder.

3. The composition of claim 1, wherein the quantity of protein include both land and sea-based proteins.

4. The composition of claim 1, wherein the quantity of probiotics is a fermented land-based organism.

5. The composition of claim 1, wherein the food composition is a paste.

6. The composition of claim 1, wherein the quantity of protein is fermented.

7. The composition of claim 1, further comprising a quantity of one or more of microalgae, plankton, fish meal, shrimp, crustacean, and algae.

8. The composition of claim 1, further comprising a quantity of vitamins and minerals; wherein the vitamins and minerals are from land-based plants.

9. The composition of claim 1, wherein the probiotic quantity includes bacteria species selected from the group of bacteria species consisting of Lactobacillus, Saccharomyces, and Baccillus; and wherein the quantity of protein is a sea-based protein selected from the group of sea-based proteins consisting of: fish, and crustacean.

10. A method of manufacturing a coral food composition, comprising the steps of:
a) providing a quantity of protein from a sea-based source or a land-based source;
b) providing a quantity of probiotics from a sea-based source or a land-based source but not the same source, land or sea, as the quantity of protein;
c) fermenting the quantity of protein with the quantity of probiotics thereby forming a fermented mixture; and
d) converting the fermented mixture into a powder or paste.

11. The method of claim 10, wherein the quantity of protein includes both land and sea-based proteins.

12. The method of claim 10, wherein the quantity of probiotics is a fermented land-based organism.

13. The method of claim 10, further comprising the step of providing a quantity of one or more of microalgae, plankton, fish meal, shrimp, crustacean, and algae.

14. The method of claim 10, further comprising providing a quantity of vitamins and minerals; wherein the vitamins and minerals are from land-based plants.

15. The method of claim 10, wherein the probiotic quantity includes bacteria species selected from the group of bacteria species consisting of Lactobacillus, Saccharomyces, and Baccillus; and wherein the quantity of protein is a sea-based protein selected from the group of sea-based proteins consisting of: fish, and crustacean.

16. A method of treating coral, comprising the steps of:
a) applying a probiotic food composition to the coral, the food composition comprising:
a1) a quantity of protein from a sea-based source; and
a2) a quantity of probiotics from a land-based source; or
a3) a quantity of protein from a land-based source; and
a4) a quantity of probiotics from a sea-based source.

17. The method of claim 16, wherein the food composition is a powder.

18. The method of claim 17, wherein the quantity of protein include both land and sea-based proteins.

19. The method of claim 18, wherein the quantity of probiotics is a fermented land-based organism, thereby introducing to the coral a non-native probiotic.

20. The method of claim 19, wherein the food composition is a paste.

21. The method of claim 20, wherein the quantity of protein is sea-based and is fermented using probiotics that are non-native to the sea, thereby forming a probiotic culture that is non-native to aquaculture environments that is able to sustain itself on sea-based proteins.

22. The method of claim 21, further comprising the step of providing a quantity of one or more of microalgae, plankton, fish meal, shrimp, crustacean, and algae.

23. The method of claim 22, further comprising the step of providing a quantity of vitamins and minerals; wherein the vitamins and minerals are from land-based plants.

24. The method of claim 23, wherein the probiotic quantity includes bacteria species selected from the group of bacteria species consisting of Lactobacillus, Saccharomyces, and Baccillus; and wherein the quantity of protein is a sea-based protein selected from the group of sea-based proteins consisting of: fish, and crustacean..
